(19) United States
(12) Patent Application Publication
(10) Pub. No.: US 2013/0116508 A1
(43) Pub. Date: May 9, 2013

(54) FLUOROSCOPY APPARATUS

(71) Applicant: OLYMPUS CORPORATION, Tokyo (JP)

(72) Inventor: Hiromi SHIDA, Tokyo (JP)

(73) Assignee: OLYMPUS CORPORATION, Tokyo (JP)

(21) Appl. No.: 13/724,815

(22) Filed: Dec. 21, 2012

Related U.S. Application Data
(63) Continuation of application No. PCT/JP2011/064651, filed on Jun. 27, 2011.

(30) Foreign Application Priority Data
Jun. 30, 2010 (JP) 2010-149052

(51) Int. Cl. A61B 1/00

(52) U.S. Cl. CPC A61B 1/00045
USPC 600/109

(57) ABSTRACT

Provided is a fluoroscopy apparatus including an illumination unit that radiates illumination light and excitation light onto a subject; a fluorescence imaging unit that acquires a fluorescence image by imaging fluorescence generated at the subject; a return-light imaging unit that acquires a return-light image by imaging return light returned from the subject; a fluorescence-image storage unit that stores the fluorescence image; a treatment input unit to which signals for starting and ending treatment performed on the subject are input; a comparison-image generating unit that generates a comparison image that enables comparison of the fluorescence image stored in the fluorescence-image storage unit when the signal for starting the treatment is input to the treatment input unit and the fluorescence image acquired when the signal for ending the treatment is input; and a display unit that displays the comparison image.

### CROSS-REFERENCE TO RELATED APPLICATIONS

This is a continuation of International Application PCT/JP2011/064,651, with an international filing date of Jun. 27, 2011, which is hereby incorporated by reference herein in its entirety. This application claims the benefit of Japanese Patent Application No. 2010-149052, the contents of which are incorporated herein by reference.

### TECHNICAL FIELD

The present invention relates to a fluoroscopy apparatus.

### BACKGROUND ART

In the related art, there are known fluorescence endoscope apparatuses that acquire a fluorescence image and a white-light image by radiating excitation light and white light onto a subject, then switch to the white-light image after an affected site in the fluorescence image is identified, allowing treatment to be performed at the affected site, such as taking a tissue sample from the affected site, while the white-light image is being observed (for example, refer to Patent Literature 1).

In such a fluorescence endoscope apparatus, adding a mark to the identified affected site in the fluorescence image and allowing the mark to remain at the position of the identified affected site after switching to a white-light image facilitates the operation during treatment of the affected site.

### CITATION LIST

### Patent Literature

{PTL 1} Publication of Japanese Patent No. 3771985

### SUMMARY OF INVENTION

An aspect of the present invention provides a fluoroscopy apparatus including an illumination unit configured to radiate illumination light and excitation light onto a subject; a fluorescence imaging unit configured to acquire a fluorescence image by imaging fluorescence generated at the subject as a result of being irradiated with the excitation light from the illumination unit and; a return-light imaging unit configured to acquire a return-light image by imaging return light returned from the subject as a result of being irradiated with the illumination light from the illumination unit and; a fluorescence-image storage unit configured to store the fluorescence image acquired by the fluorescence imaging unit; a treatment input unit to which input signals for starting and ending treatment performed on the subject are input; a comparison-image generating unit configured to generate a comparison image that enables comparison of the fluorescence image stored in the fluorescence-image storage unit when the signal for starting the treatment is input to the treatment input unit and the fluorescence image acquired when the signal for ending the treatment is input to the treatment input unit; and a display unit configured to display the comparison image generated by the comparison-image generating unit.

According to an aspect of the present invention, a display switching unit configured to switch and display, on the display unit, the return-light image acquired by the return-light imaging unit, the fluorescence image acquired by the fluorescence imaging unit, and the comparison image generated by the comparison-image generating unit may be further included, and the display switching unit, while in a state in which the fluorescence image is displayed on the display unit, may switch the display image on the display unit to the return-light image when the signal for starting the treatment is input to the treatment input unit and, while in a state in which the return-light image is displayed on the display unit, may switch the display image on the display unit to the comparison image when the signal for ending the treatment is input to the treatment input unit.

In addition, according to an aspect of the present invention, the comparison-image generating unit may generate a comparison image in which the fluorescence image stored in the fluorescence-image storage unit when the signal for starting the treatment is input to the treatment unit and the fluorescence image acquired when the signal for ending the treatment is input to the treatment unit are placed side-by-side.

In addition, according to an aspect of the present invention, the comparison-image generating unit may generate a difference image, as the comparison image, by calculating the difference between the fluorescence image stored in the fluorescence-image storage unit when the signal for starting the treatment is input to the treatment input unit and the fluorescence image acquired when the signal for ending the treatment is input to the treatment input unit.

In addition, according to an aspect of the present invention, a return-light-image storage unit configured to store the return-light image when the signal for starting the treatment is input to the treatment input unit and a positional-displacement calculating unit configured to calculate positional displacement between the return-light image acquired when the signal for ending the treatment is input to the treatment input unit and the return-light image stored in the return-light-image storage unit may be further included, and the comparison-image generating unit may generate the difference image after performing alignment of the fluorescence images by using the positional displacement calculated by the positional-displacement calculating unit.

In addition, according to an aspect of the present invention, a peak extracting unit configured to extract a peak region in which the fluorescence intensity is the highest in the fluorescence image stored in the fluorescence-image storage unit when the signal for starting the treatment is input to the treatment input unit may be further included, and the comparison-image generating unit may generate a comparison image in which the peak region extracted by the peak extracting unit is superposed on the difference image.

### BRIEF DESCRIPTION OF DRAWINGS

FIG. 1 is an overall configuration diagram illustrating a fluoroscopy apparatus according to a first embodiment of the present invention.

FIG. 2 is a diagram illustrating an example of a comparison image generated by the fluoroscopy apparatus in FIG. 1.

FIG. 3 is a flow chart illustrating the observation procedure using the fluoroscopy apparatus in FIG. 1.

FIG. 4 is a diagram illustrating the observation procedure using the fluoroscopy apparatus in FIG. 1, where each of the following are illustrated: (a) a white-light image; (b) a fluorescence image; (c) a fluorescence image to which a mark is added; (d) a white-light image in which the mark remains; (e) treatment using the white-light image; and (f) a comparison image.

FIG. 5 is an overall configuration diagram illustrating a fluoroscopy apparatus according to a second embodiment of the present invention.

FIG. 6 is an overall configuration diagram illustrating a modification of the fluoroscopy apparatus in FIG. 5.

FIG. 7 is a diagram illustrating a peak region in a fluorescence image acquired by the fluoroscopy apparatus in FIG. 6.

### DESCRIPTION OF EMBODIMENTS

A fluoroscopy apparatus 1 according to a first embodiment of the present invention will be described below with reference to the drawings.

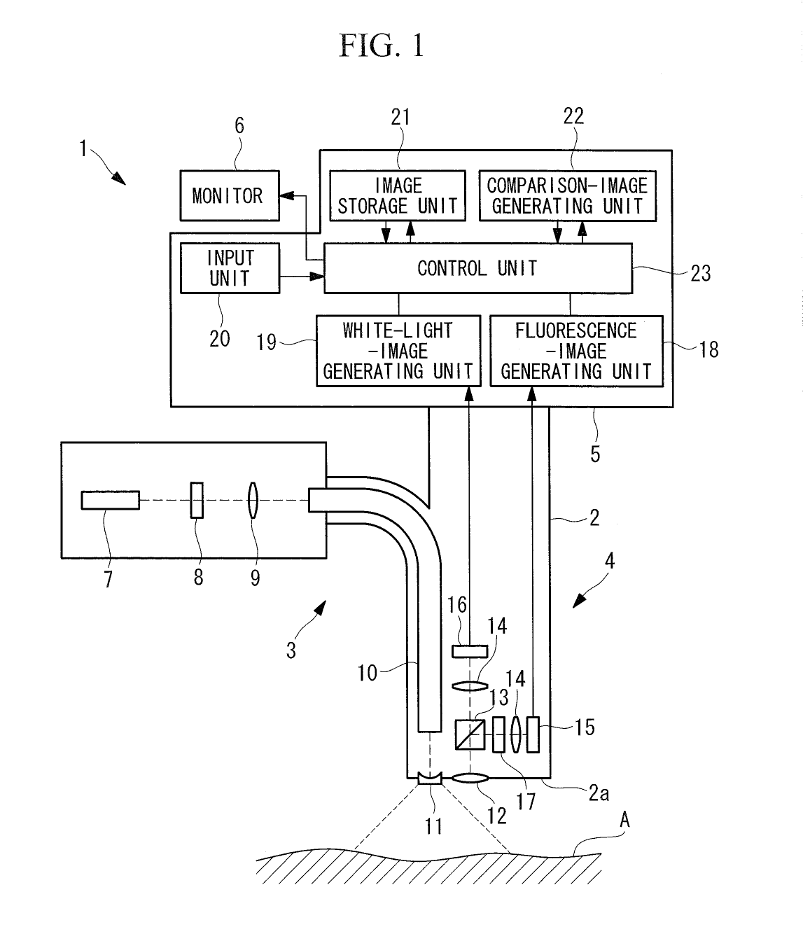

As illustrated in FIG. 1, the fluoroscopy apparatus 1 according to this embodiment is an endoscope apparatus and includes an insertion part 2 that is inserted into a body cavity, an illumination unit 3 that radiates white light (illumination light) and excitation light to biological tissue (hereinafter, referred to as “subject”) A disposed so as to oppose the tip face 2a of the insertion part 2, an image acquisition unit 4 that acquires a fluorescence-image signal and a white-light image signal of the subject A, an image processing unit 5 that processes the image signals acquired by the image acquisition unit 4, and a monitor (display unit) 6 that displays an image generated by the image processing unit 5.

The illumination unit 3 includes a xenon lamp 7, a filter 8, a coupling lens 9, a light-guiding fiber 10, and an illumination optical system 11. The filter 8 has transmittance characteristics whereby light in the wavelength band of white light and excitation light (for example, 400 to 750 nm) is transmitted and light in other wavelength bands is blocked.

The coupling lens 9 focuses the white light and excitation light transmitted through the filter 8 to make it incident on the end portion of the light-guiding fiber 10. The light-guiding fiber 10 is disposed along the entire length of the insertion part 2, from the base to the tip, and guides the white light and excitation light to the tip face 2a of the insertion part 2. The illumination optical system 11 spreads out the white light and the excitation light guided through the light-guiding fiber 10 to radiate the light onto the subject A.

The image acquisition unit 4 includes an objective lens 12 that is disposed at the tip of the insertion part 2 and collects fluorescence generated at the subject A and returning white light from the subject A, a dichroic mirror 13 that splits the light collected by the objective lens 12 into fluorescence and white light, a focusing lens 14 that focuses each of the fluorescence and the white light split at the dichroic mirror 13, a fluorescence CCD (fluorescence imaging unit) 15 that images the fluorescence focused by the focusing lens 14, and a white-light CCD (return-light imaging unit) 16 that images the white light focused by the focusing lens 14. In the drawings, reference numeral 17 represents an excitation-light cut filter that stops excitation light from being incident on the fluorescence CCD 15.

The image processing unit 5 includes a fluorescence-image generating unit 18 that processes the fluorescence-image signal acquired by the fluorescence CCD 15 and generates a fluorescence image, a white-light-image generating unit 19 that processes the white-light image signal acquired by the white-light CCD 16 and generates a white-light image, an input unit (treatment input unit) 20 that receives a command signal input from an external unit, an image storage unit 21 in which the fluorescence image is stored, a comparison-image generating unit 22 that generates a comparison image, and a control unit (display switching unit) 23 that receives inputs from the fluorescence-image generating unit 18, the white-light-image generating unit 19, and the input unit 20 and controls the image storage unit 21, the comparison-image generating unit 22, and the monitor 6.

The input unit 20 is input means, such as a push button that inputs a command signal when the operator, such as a doctor, starts treatment and ends treatment of the subject A.

When a command signal for starting treatment is input from the input unit 20, the control unit 23 sends the fluorescence image acquired at that point in time to the image storage unit 21 for storage.

In addition, when a command signal for ending the treatment is input from the input unit 20, the control unit 23 sends the fluorescence image of the subject A after treatment, acquired at that point in time, and the fluorescence image at the start of treatment, stored in the image storage unit 21, to the comparison-image generating unit 22 to generate a comparison image.

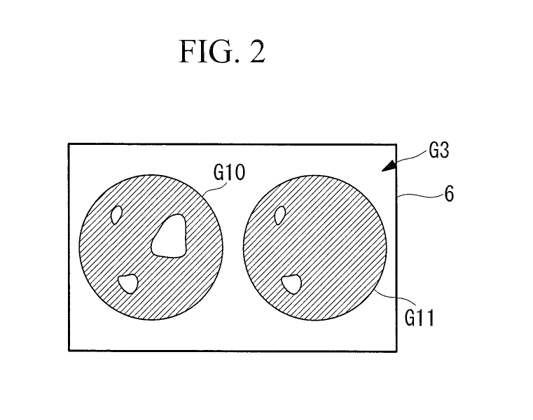

The comparison-image generating unit 22 generates, for example, as illustrated in FIG. 2, a comparison image G3 in which a fluorescence image G10 at the start of the treatment and a fluorescence image G11 at the end of the treatment are placed side-by-side and inputs this to the control unit 23.

In addition, the control unit 23 switches among the fluorescence image, the white-light image, and the comparison image and displays these on the monitor 6.

For example, the control unit 23 displays a fluorescence image to allow the operator, such as a doctor, to observe the condition of a lesion site on the fluorescence image and also stores a mark when a mark is added by the operator to a high luminance region, etc., in the fluorescence image.

Then, the control unit 23 switches the display on the monitor 6 from the fluorescence image to the white-light image when a command signal for starting the treatment is input from the input unit 20, and after switching, the mark added to the fluorescence image remains on the white-light image.

The operator performs treatment on the region to which the mark is added while confirming the surface condition of the subject A in the white-light image, and upon ending the treatment, a command signal for ending the treatment is input from the input unit 20.

When a command signal for ending the treatment is input to the input unit 20, instead of the white-light image displayed on the monitor 6, the control unit 23 displays the comparison image input from the comparison-image generating unit 22.

The operation of the fluoroscopy apparatus 1 according to this embodiment having such a configuration will be described below.

To perform fluoroscopy of the subject A using the fluoroscopy apparatus 1 according to this embodiment, fluorescent probes that preferentially accumulate in a lesion site, etc. are added to the subject A while the insertion part 2 is inserted into the body cavity such that the tip face 2a opposes the subject A.

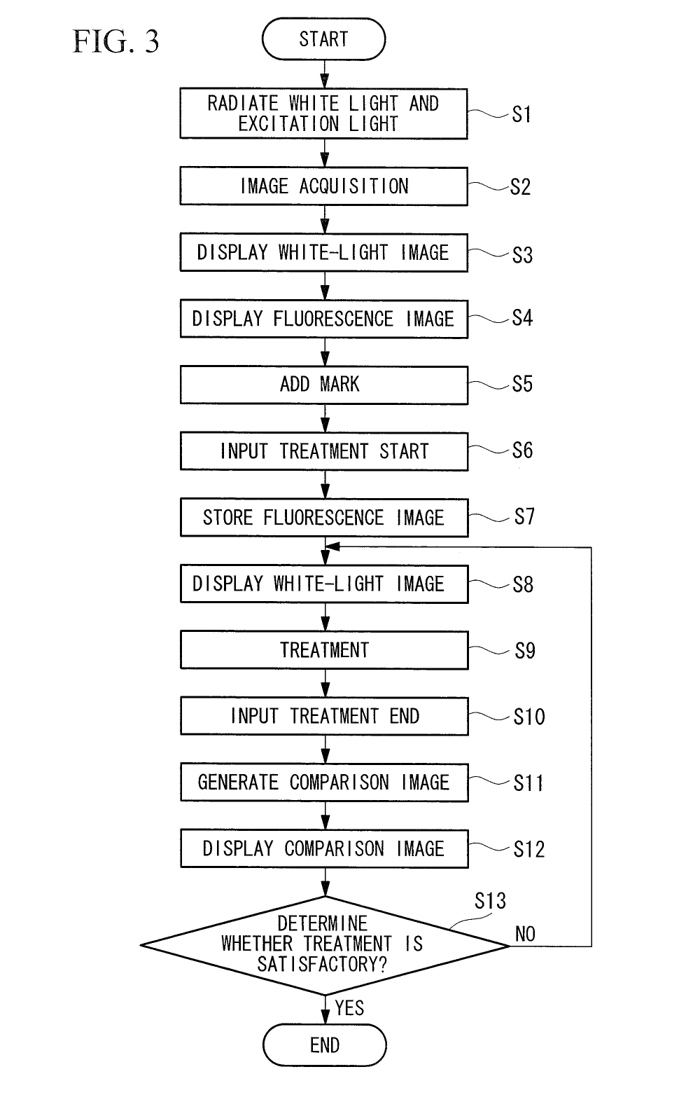

Then, as illustrated in FIG. 3, the illumination unit 3 is operated to radiate white light and excitation light onto the subject A from the illumination optical system 11 at the tip of the insertion part 2 through the light-guiding fiber 10. By radiating excitation light, the fluorescent probes in the subject A are excited, generating fluorescence. In addition, as a result of radiating white light, the reflected light of the white light from the surface of the subject A returns to the tip face 2a side of the insertion part 2.

The fluorescence and the white light incident on the tip face 2a side of the insertion part 2 are collected by the objective lens 12, split by the dichroic mirror 13, and imaged by the fluorescence CCD 15 and the white-light CCD 16, respectively. The fluorescence image signal output from the fluorescence CCD 15 is sent to the fluorescence-image generating unit 18 of the image processing unit 5, where the fluorescence image G1 is generated. Also, the white-light-image signal output from the white-light CCD 16 is sent to the white-light-image generating unit 19 of the image processing unit 5, where the white-light image G2 is generated (Step S2).

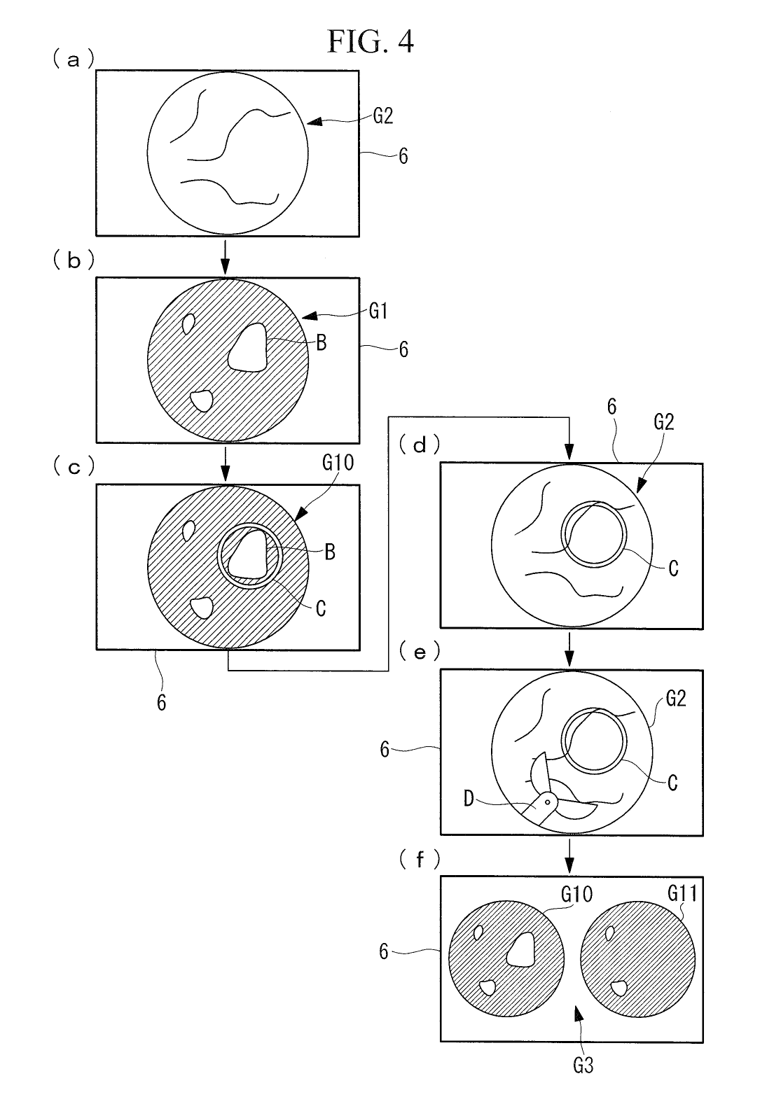

First, as illustrated in FIG. 4(a), the control unit 23 displays the white-light image G2 on the monitor 6 (Step S3). Then, the operator manipulates the insertion part 2 while observing the surface condition of the subject A in the white-light image G2 on the monitor 6 and, when approaching a site suspected of being a lesion site, switches the display on the monitor 6 to the fluorescence image G1, as illustrated in FIG. 4(b) (Step S4).

The operator searches for a lesion site B, which is a region with high fluorescence brightness, while viewing the fluorescence image G1 displayed on the monitor 6, and, upon discovery, adds a mark C to the region, as illustrated in FIG. 4(c) (Step S5). The addition of the mark C is performed on the monitor 6 by a mouse, etc., which is not shown, and is stored in the control unit 23. Then, using the input unit 20, the operator performs input for starting treatment with (Step S6).

When a signal for starting treatment is input to the input unit 20, the control unit 23 sends the fluorescence image G10 displayed on the monitor 6 at that point in time to the image storage unit 21 for storage (Step S7) while displaying the white-light image G2 on the monitor 6, instead of the fluorescence image G1, as illustrated in FIG. 4(d). At this time, the mark C added on the fluorescence image G1 and stored in the control unit 23 remains on the white-light image G2.

While confirming the surface condition of the subject A in the white-light image G2, the operator performs treatment such as excising tissue using, for example, a treatment tool D, as illustrated in FIG. 4(e), in the region to which the mark C is added. Then, once the treatment ends, using the input unit 20, the operator performs input for ending the treatment (Step S10).

When the signal for ending the treatment is input to the input unit 20, the control unit 23 sends the fluorescence image G11 acquired at that point in time and the fluorescence image G10 stored in the image storage unit 21 to the comparison-image generating unit 22, which generates a comparison image G3 (Step S11). The comparison-image generating unit 22 generates the comparison image G3 in which the two fluorescence images G10 and G11 are placed side-by-side and sends this to the control unit 23. Then, as illustrated in FIG. 4(f), the control unit 23 displays the comparison image G3 sent from the comparison-image generating unit 22 in place of the white-light image G2 displayed on the monitor 6 (Step S12).

In this way, the operator can readily confirm the change in the fluorescence images G10 and G11 acquired before and after the treatment by directly comparing the fluorescence images G10 and G11 acquired before and after the treatment in the comparison image G3 displayed on the monitor 6. If sufficient treatment has not been performed, the procedures from Step S8 are repeated (Step S13).

In this way, the fluoroscopy apparatus 1 according to this embodiment has advantages in that it is possible to readily confirm whether the treatment performed while confirming the surface condition in the white-light image G2 has been sufficiently performed by displaying the fluorescence images G10 and G11 acquired before and after the treatment side-by-side, observation can be efficiently performed, and the precision of the treatment can be enhanced.

On the other hand, the fluorescence endoscope apparatus in the related art has a drawback in that, although the position of an affected site can be identified even after switching to the white-light image, it was difficult to confirm whether desired treatment has been performed.

The fluoroscopy apparatus 1 according to this embodiment enables the result of the treatment performed at an affected site to be readily confirmed.

Next, a fluoroscopy apparatus 30 according to a second embodiment of the present invention will be described below with reference to the drawings.

In the description of the fluoroscopy apparatus 30 according to this embodiment, the portions that are the same as those in the configuration of the fluoroscopy apparatus 1 according to the first embodiment described above are designated with the same reference numerals, and descriptions thereof are omitted.

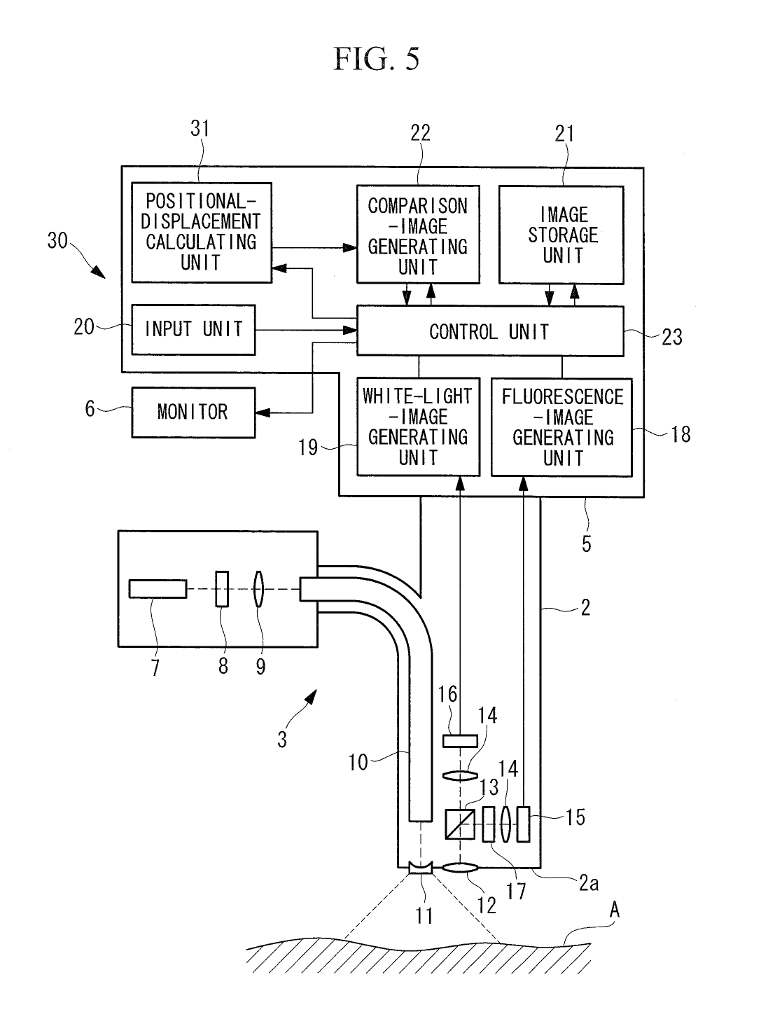

As illustrated in FIG. 5, the fluoroscopy apparatus 30 according to this embodiment differs from the fluoroscopy apparatus 1 according to the first embodiment in the operation of the control unit 23, in that a positional-displacement calculating unit 31 for calculating the displacement of the white-light images G2 acquired before and after treatment is provided, and in that, as the comparison image G3, the comparison-image generating unit 22 generates a difference image of the fluorescence images G10 and G11 acquired before and after the treatment.

As illustrated in FIG. 5, when a signal for starting treatment is input to the input unit 20, the control unit 23 sends the fluorescence image G10 and the white-light image acquired at that point in time to the image storage unit 21 for storage. In addition, when a signal for ending the treatment is input to the input unit 20, the control unit 23 sends the fluorescence image G11 acquired after the treatment, acquired at that point in time, and the fluorescence image G10 acquired before the treatment, stored in the image storage unit 21, to the comparison-image generating unit 22 and sends the white-light image acquired after the treatment and the white-light image acquired before the treatment, stored in the image storage unit 21, to the positional-displacement calculating unit 31.

The positional-displacement calculating unit 31 receives the white-light image acquired before the treatment, stored in the image storage unit 21, and the white-light image acquired after the treatment, acquired when the signal for ending the treatment is input to the input unit 20, and calculates the positional displacement between these white-light images. Specifically, by extracting characteristic points in each white-light image (for example, a geometric characteristic point, a chromatic characteristic point, etc.) and determining the moving directions and distances of the corresponding extracted characteristic points, the directions and distances in which the field of view has shifted before and after the treatment are determined.

The comparison-image generating unit 22 receives the fluorescence image G10 acquired before the treatment, stored in the image storage unit 21, the fluorescence image G11 acquired after the treatment, acquired when the signal for ending the treatment is input to the input unit 20, and the positional displacement calculated by the positional-displacement calculating unit 31.

At the comparison-image generating unit 22, first, alignment processing of the two fluorescence images G10 and G11 is performed. That is, since the fields of view of the simultaneously acquired white-light image G2 and fluorescence image G1 match, the fluorescence images G10 and G11 are also displaced by the same positional displacement in the same direction, as calculated using the white-light images acquired before and after the treatment. Thus, by relatively moving the two fluorescence images G10 and G11 by the inputted positional displacement, alignment can be readily performed.

Next, at the comparison-image generating unit 22, the difference between the two aligned fluorescence images G10 and G11 is calculated. The difference image, which is the comparison image G3 generated by the comparison-image generating unit 22, is displayed on the monitor 6.

The thus-configured fluoroscopy apparatus 30 according to this embodiment is advantageous in that, since the comparison image G3, which is composed of a difference image representing the difference between the fluorescence images G10 and G11 acquired before and after the treatment, is displayed on the monitor 6, minute differences in the fluorescence image G1, which cannot be determined by displaying them side-by-side, can also be clearly confirmed. In particular, since the fluorescence images G10 and G11 acquired before and after the treatment are aligned using the white-light images acquired before and after the treatment, it is possible to accurately display only the areas that have changed in the fluorescence images G10 and G11 acquired before and after the treatment.

Therefore, the change in the fluorescence images G10 and G11 acquired before and after the treatment can be precisely extracted as a difference image.

In this embodiment, a difference image is displayed on the monitor 6 as the comparison image G3. Instead, however, the comparison image G3, which is composed of a difference image, may be displayed beside the fluorescence image G11 acquired after treatment, or the comparison image G3, which is composed of a difference image, may be displayed superposed on the fluorescence image G11 acquired after the treatment.

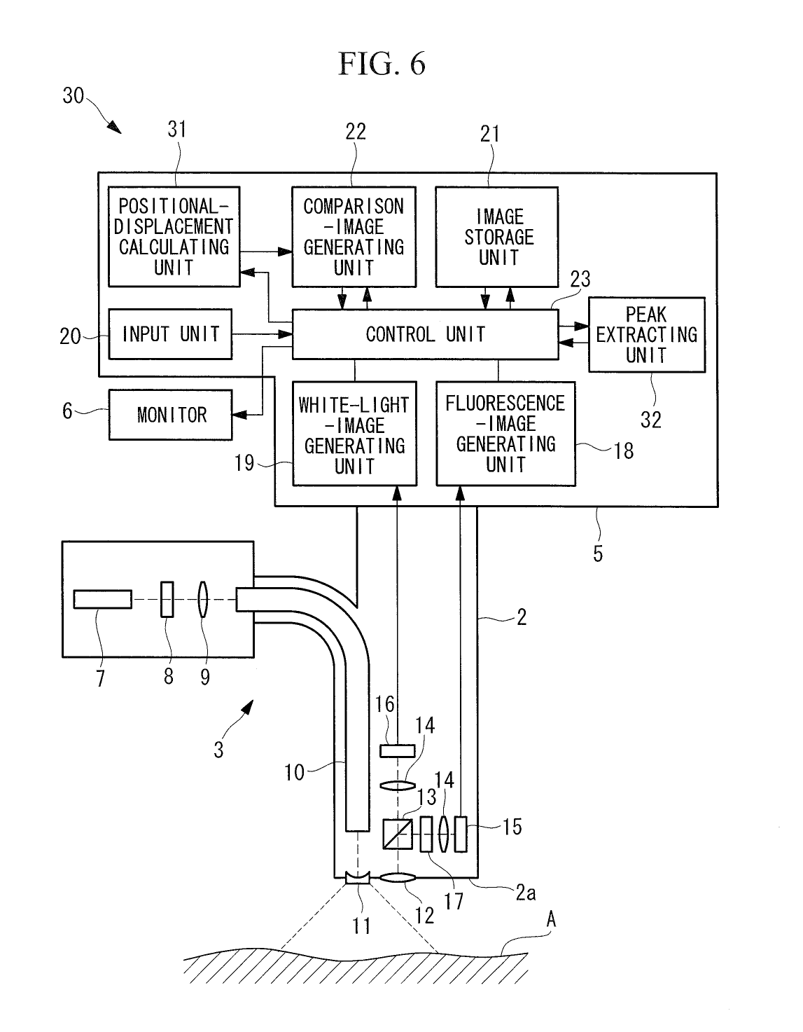

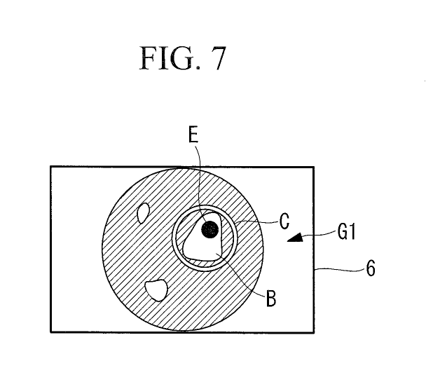

In addition, as illustrated in FIG. 6, a peak extracting unit 32 that extracts a peak region E including the pixel having the highest fluorescence intensity in the fluorescence image G10 acquired before the treatment may be provided, and the extracted peak region E may be stored in the control unit 23 to display the peak region E together with the mark C when displaying the white-light image G2, as illustrated in FIG. 7, or the peak region E may be displayed together with the comparison image G3.

Furthermore, it is determined whether the region including the difference of the comparison image G3 and the peak region E match; if the peak region E and the region including the difference match, in order to indicate that the treatment has been performed in the correct region, the color of the screen display may be changed or a message such as “correct treatment performed” may be displayed.

By doing so, it is possible to confirm, at a glance, whether appropriate treatment has been performed on the peak region E in which the fluorescence intensity is the highest.

In addition, in this embodiment, the alignment of the fluorescence images G10 and G11 acquired before and after the treatment is performed using the positional displacement calculated from the corresponding white-light images acquired before and after the treatment. Instead, however, the characteristic points of the fluorescence images G10 and G11 themselves may be extracted to perform alignment processing.

In addition, reflected white-light has been given as an example of the return light. Instead, however, any other light, such as near infrared light and autofluorescence, may be used as the return light.

### REFERENCE SIGNS LIST

A subject
E peak region
G1, G10, G11 fluorescence image
G2 white-light image (return-light image)
G3 comparison image
1, 30 fluoroscopy apparatus
3 illumination unit
6 monitor (display unit)
15 fluorescence CCD (fluorescence imaging unit)
16 white-light CCD (return-light imaging unit)
20 input unit (treatment input unit)
21 image storage unit (fluorescence-image storage unit, return-light storage unit)
22 comparison-image generating unit
23 control unit (display switching unit)
31 positional-displacement calculating unit
32 peak extracting unit

1. A fluoroscopy apparatus comprising:
an illumination unit configured to radiate illumination light and excitation light onto a subject;
a fluorescence imaging unit configured to acquire a fluorescence image by imaging fluorescence generated at the subject as a result of being irradiated with the excitation light from the illumination unit;
a return-light imaging unit configured to acquire a return-light image by imaging return light returned from the subject as a result of being irradiated with the illumination light from the illumination unit;
a fluorescence-image storage unit configured to store the fluorescence image acquired by the fluorescence imaging unit;
a treatment input unit to which input signals for starting and ending treatment performed on the subject are input;
a comparison-image generating unit configured to generate a comparison image that enables comparison of the fluorescence image stored in the fluorescence-image storage unit when the signal for starting the treatment is input to the treatment input unit and the fluorescence image acquired when the signal for ending the treatment is input to the treatment input unit; and
a display unit configured to display the comparison image generated by the comparison-image generating unit.

2. The fluoroscopy apparatus according to claim 1, further comprising:
a display switching unit configured to switch between and display, on the display unit, the return-light image acquired by the return-light imaging unit, the fluorescence image acquired by the fluorescence imaging unit, and the comparison image generated by the comparison-image generating unit,
wherein the display switching unit, while in a state in which the fluorescence image is displayed on the display unit, switches the display image on the display unit to the return-light image when the signal for starting the treatment is input to the treatment input unit and, while in a state in which the return-light image is displayed on the display unit, switches the display image on the display unit to the comparison image when the signal for ending the treatment is input to the treatment input unit.

3. The fluoroscopy apparatus according to claim 1, wherein the comparison-image generating unit generates the comparison image in which the fluorescence image stored in the fluorescence-image storage unit when the signal for starting the treatment is input to the treatment input unit and the fluorescence image acquired when the signal for ending the treatment is input to the treatment input unit are placed side-by-side.

4. The fluoroscopy apparatus according to claim 1, wherein the comparison-image generating unit generates a difference image, as the comparison image, by calculating the difference between the fluorescence image stored in the fluorescence-image storage unit when the signal for starting the treatment is input to the treatment input unit and the fluorescence image acquired when the signal for ending the treatment is input to the treatment input unit.

5. The fluoroscopy apparatus according to claim 4, further comprising:
a return-light-image storage unit configured to store the return-light image when the signal for starting the treatment is input to the treatment input unit; and
a positional-displacement calculating unit configured to calculate positional displacement between the return-light image acquired when the signal for ending the treatment is input to the treatment input unit and the return-light image stored in the return-light-image storage unit,
wherein the comparison-image generating unit generates the difference image after performing alignment of the fluorescence images by using the positional displacement calculated by the positional-displacement calculating unit.

6. The fluoroscopy apparatus according to claim 4, further comprising:
a peak extracting unit configured to extract a peak region having a highest fluorescence intensity in the fluorescence image stored in the fluorescence-image storage unit when the signal for starting the treatment is input to the treatment input unit,
wherein the comparison-image generating unit generates a comparison image in which the peak region extracted by the peak extracting unit is superposed on the difference image.

7. The fluoroscopy apparatus according to claim 2, wherein the comparison-image generating unit generates the comparison image in which the fluorescence image stored in the fluorescence-image storage unit when the signal for starting the treatment is input to the treatment input unit and the fluorescence image acquired when the signal for ending the treatment is input to the treatment input unit are placed side-by-side.

8. The fluoroscopy apparatus according to claim 2, wherein the comparison-image generating unit generates a difference image, as the comparison image, by calculating the difference between the fluorescence image stored in the fluorescence-image storage unit when the signal for starting the treatment is input to the treatment input unit and the fluorescence image acquired when the signal for ending the treatment is input to the treatment input unit.

9. The fluoroscopy apparatus according to claim 8, further comprising:
a return-light-image storage unit configured to store the return-light image when the signal for starting the treatment is input to the treatment input unit; and
a positional-displacement calculating unit configured to calculate positional displacement between the return-light image acquired when the signal for ending the treatment is input to the treatment input unit and the return-light image stored in the return-light-image storage unit,
wherein the comparison-image generating unit generates the difference image after performing alignment of the fluorescence images by using the positional displacement calculated by the positional-displacement calculating unit.

10. The fluoroscopy apparatus according to claim 8, further comprising:
a peak extracting unit configured to extract a peak region having a highest fluorescence intensity in the fluorescence image stored in the fluorescence-image storage unit when the signal for starting the treatment is input to the treatment input unit,
wherein the comparison-image generating unit generates a comparison image in which the peak region extracted by the peak extracting unit is superposed on the difference image.
